(19) United States
(12) Patent Application Publication
(10) Pub. No.: US 2008/0085871 A1
(43) Pub. Date: Apr. 10, 2008

(54) Novel polysaccharide pro-drug 5-fluorouracil (5-FU) with enhanced target specificity for colorectal cancer and its preparation methods

(76) Inventors: Joemy C. Tam, Medford, NY (US); Qi Bing Mei, Xi'an (CN); Li Liu, Xi'an (CN); Qing wei Wang, Xi'an (CN)

Correspondence Address: Joemy C. Tam, Suite 2, Bldg. 5, 3235 Route 112, Medford, NY, 11763, US

(21) Appl. No.: 11/657,753

(22) Filed: Sep. 17, 2007

(30) Foreign Application Priority Data
Feb. 23, 2006 (CN) 200610041838.0

(51) Int. Cl. A61K 31/7072; A61P 35/00; C07H 1/00; C07H 19/06; C07H 3/00

(52) U.S. Cl. 514/50; 536/123.1; 536/55.1; 536/55.3

(57) ABSTRACT

This invention describes a novel polysaccharide prodrug of 5-fluorouracil (5-FU) with enhanced target specificity for colorectal cancer treatment, and its preparation methods. The prodrug is synthesized by chemically linking anti-cancer drug 5-fluorouracil (5-FU) with a specially selected polysaccharide with molecular weight of 10⁵˜10⁷ Da containing galactose residues. Its distinctive characteristics are that it is a prodrug synthesized by chemically linking polysaccharides with 5-FU through different bridge links for the targeted treatment of colorectal cancer; that the polysaccharides in the chemical compound contain galactose residues; and that these polysaccharides are prepared from natural gums or plant materials. Due to these unique characteristics, as an oral preparation, the polysaccharide component of this novel prodrug can protect the active agent 5-FU from absorption (or metabolism) in the upper gastrointestinal tract and deliver a high concentration of the 5-FU to the colorectal area. Upon reaching the colorectal area, the 5-FU-galactose portion of the prodrug will bind to galectin-3, a-galactoside-binding protein implicated in tumor progression by interactions with its ligands, such as TF (Thomsen-Friedenreich, Galb3GalNAc), Tn (GalNAcaThr/Ser), and Sialy-Tn with galactose residues, which are highly expressed among colorectal cancer cells. Finally, the active 5-FU component will be released locally from the polysaccharide via enzymatic hydrolysis from the local bacterial flora, allowing it to actively kill the colorectal cancer cells. In summary, this novel target-specific prodrug can enhance the selectivity of 5-FU and increase its therapeutic effects in the treatment of colorectal cancer. In addition, with this enhanced target specificity, it is possible to maximize the 5-FU efficacy in cancer patients by having either less toxicity with the same or higher therapeutic dose, and/or administer a lower dosage (if so desired) to achieve the same therapeutic effects, but with much less toxicity. Multiple examples of various approaches to synthesize this novel prodrug are enclosed herein along with several animal model experiments to substantiate the claims as stated above.

Throughout this application, references are made to various publications. Disclosures of these publications in their entireties are hereby incorporated by reference into this application to more fully describe the state of the art to which this invention pertains.

### FIELD OF THE INVENTION

This invention involves drugs for the treatment of colorectal cancer and their preparation methods, which mainly concern a kind of prodrug composed of the anticancer drug 5-fluorouracil (5-FU) and polysaccharides containing galactoses and its preparation methods.

### BACKGROUND OF THE INVENTION

Colorectal cancer is the third most common kind of cancer and the second leading cause of death among cancers in the people of the Western world. At present, there are no fully effective treatments available. Part of the reasons why this is the case is that for the anti-cancer drug to reach the colorectal area, a systemic administration is usually required. However, because of the lack of selectivity, severe systemic toxicities (such as bone marrow suppression, mucositis, etc) will result, which makes the patients so sick that it will limit an effective dose a patient can receive.

5-fluorouracil (5-FU) is a well-established chemotherapeutic agent. It is a pyrimidine analog and belongs to the class of anti-metabolites. It is effective in colorectal and many gastrointestinal malignancies as well as breast and skin cancers. Once administered to a patient, 5-FU tends to distribute randomly throughout the body with low selectivity and concentration in the lumina of the colorectal tissue, resulted in significant toxicities. If an appropriate large molecular carrier is chemically linked to the 5-FU with specificity for colorectal cancer cells, this will enhance the therapeutic index as well as allow the dosage of the drug to be reduced (if so desired) to minimize toxicities. In addition, with the higher specificity and lower toxicity of such prodrug disclosed herein, the oncologist could also titrate the dosage to the level that a patient could maximally tolerate, thus maximizing the efficacy and the likelihood of a positive outcome for the patient.

Large molecule carriers include ethylene or acrylic acid polymers, polysaccharides, hydroxyl-acid polymers, and amino acid polymers, etc. Ouchi and others reported 4 kinds of conjugations composed of 5-FU and chitosan through ester linkage, amino formamide, amide linkage and ether linkage (Ouchi et al, 1992). Ohya and coworkers reported a conjugate of 5-FU and 6-O-carboxymethyl chitosan, which has an inhibitory effect on P388 lymphoid leucosis (Ohya et al 1992, 1993). Fan and colleagues composed a co-polymer of lactic acid-phosphate ester with 5-FU as the model drug in the side chain and the copolymer of lactic acid-phosphate ester as the large molecule prodrug carrier, which shows lower toxicity and better antitumor activity (Fan et al, 1985; Luo et al, 1994). Zhu and others linked 5-FU with poly-L-(2-ethoxyl)-asparagine to make a large molecule prodrug, which shows improved drug release effect in rabbits and maintains a steady plasma concentration during release (Zhu et al, 2003).

The literature above described several possible means to prepare 5-FU prodrugs. However, the scope of those compounds and methods focus on drug release with no disclosure or guidance relating to preparing a prodrug that shows target specificity for 5-FU delivery to colorectal cancer cells. For example, a slow-release preparation only prolongs the release time of the drug in the body but cannot direct the drug specifically to the target tissue in the colorectal area. Therefore, a slow-release preparation is completely different from a targeting preparation in pharmacokinetics, efficacy, and safety profile. Therefore, the current invention is not encompassed by any other preparations known in the art.

Over the years, there are also a number of patents applied to other 5-FU or related subjects. The following is a list briefly describing these various patents, which have been applied to 5-FU or related matters in various aspects:
U.S. Pat. No. 4,605,738, issued to Kamata et al in 1986, discloses a process of producing 1-phthalidyl-5-fluorouracil derivative as an anticancer agent.
U.S. Pat. No. 4,622,325, issued to Fujii et al in 1986, discloses a formulation comprises a combination of 1-n-hexylcarbamoyl-5-fluorouracil and a uracil salt.
U.S. Pat. No. 4,631,342, issued to Umemoto et al in 1986, discloses a process for producing 5-fluorouracil using an aqueous phosphoric acid solution as a solvent.
U.S. Pat. Nos. 4,650,801 and 4,652,570, issued to Fujii et al both in 1987, discloses a formulation comprises a combination of a 5-fluorouracil derivative and a uracil derivative.
U.S. Pat. No. 4,704,393, issued to Wakabayashi et al in 1987, discloses a compound as 1-substituted 5-fluorouracil for use in inhibiting platelets aggregation.
U.S. Pat. No. 4,719,213, issued to Fujii et al in 1988, discloses a formulation comprises a combination of a 5-fluorouracil prodrug [as either 1,3-bis(2-tetrahydrofuryl)-5-fluorouracil or 3-(2-tetrahydrofuryl)-5-fluorouracil] and a uracil salt.
U.S. Pat. No. 4,757,139, issued to Kawaguchi et al in 1988, discloses a 5-fluoro-2′-deoxyuridine derivative with anti-tumor effect in low doses.
U.S. Pat. No. 4,810,790, issued to Fujii et al in 1989, discloses 5-fluorouracil derivatives of the formula: ##STR1## wherein R.sup.1 is a fluorine-containing C.sub.1-C.sub.10 organic group which optionally contains sulfur, oxygen and/or nitrogen, with usage as a carcinostatic substance.
U.S. Pat. Nos. 4,864,021 and 4,983,609, issued to Fujii in 1989 and 1991 respectively, discloses 5-fluorouracil derivative residue of the formula ##STR1## or ##STR2## using various chemical subgroups as substitutes for the R-component, which can be converted to 5-fluorouracil in vivo and is linked to the carbonyl part by an ester or amide linkage.
U.S. Pat. No. 4,914,105, issued to Fujii et al in 1990, discloses a formulation comprises a combination of a 5-fluorouracil derivative and a uracil derivative.
U.S. Pat. No. 5,032,680, issued to Kawai et al in 1991, discloses a 2′-deoxy-5-fluorouridine derivative, which exhibits anti-tumor activities with purported lower toxicity.
U.S. Pat. No. 5,047,521, issued to Fujii et al in 1991, discloses 5-fluorouracil derivatives represented by the formula ##STR1## or ##STR2## using various chemical subgroups as substitutes for the R-component, with the provision that R.sup.1 and R.sup.2 are not hydrogen atoms or specific acyl groups at the same time.
U.S. Pat. No. 5,049,551, issued to Koda et al in 1991, discloses a 5-fluorouracil derivative represented by the formula ##STR1##, ##STR2##, and ##STR3## wherein various chemical subgroups are used as substitutes for the R-component.
U.S. Pat. No. 5,077,055, issued to Muller et al in 1991, discloses a topical therapeutic system comprising 5-fluorouracil.
U.S. Pat. No. 5,089,503, issued to Johnson in 1992, discloses a temperature stable 5-fluorouracil formulation.
U.S. Pat. No. 5,116,600, issued to Fujii et al in 1992, discloses a composition and method for inhibiting inflammation caused by non-parenteral administration of 5-fluorouracil type compounds.
U.S. Pat. Nos. 5,457,187 and 5,663,321, issued to Gmeiner et al in 1995 and 1997, respectively, discloses oligonucleotides containing 5-fluorouracil exhibit antitumor activity and the synthesis and utilization method.
U.S. Pat. No. 5,496,810, issued to Schwartz in 1996, discloses a method of treating malignancies using a combination of 5-fluorouracil, alpha-interferon, and pyrimidine deoxyribonucleoside.
U.S. Pat. No. 5,610,160, issued to Sloan et al in 1997, discloses a topical 5-fluorouracil prodrug formulation and its preparation method.
U.S. Pat. No. 5,614,505, issued to Gmeiner et al in 1997, discloses a homo-oligomeric prodrug of 5-fluorouridine (5-FU) and 5-fluorodeoxyuridine, which is used as a polymeric drug delivery system for the production of FdUMP, the active inhibitor of thymidylate synthase.
U.S. Pat. Nos. 5,627,187 and 5,817,666, issued to Katz in 1997 and 1998, respectively, discloses a dermatologic formulary preparation of 5-fluorouracil with alpha hydroxy carboxylic acid for the treatment of actinic kerotoses.
U.S. Pat. No. 5,676,973, issued to Levin in 1997, discloses a topical formulation combining 5-fluorouracil and Live Yeast Cell Derivative (LYCD) for medical use.
U.S. Pat. No. 5,808,049, issued to Yamazaki, et al in 1998, discloses a method for preparing a stereospecific 5-FU ester compound that is resistant to decomposition in blood, but is quickly hydrolyzed in cancer cells.
U.S. Pat. No. 5,843,917, issued to Boyd et al in 1998, discloses compounds comprising 5-fluorouracil or 5-fluorouridine covalently linked to 5-ethynyluracil, 5-ethynyluridine or 5-propynyluracil for pharmaceutical use.
U.S. Pat. No. 6,403,569, issued to Achterrath in 2002, discloses a method for treating cancer using camptothecin derivatives and 5-fluorouracil.
U.S. Pat. No. 6,670,335, issued to Singh et al in 2003, discloses a topical oil-in-water emulsion formulation contains 5-fluorouracil and 5-fluorouracil impregnated in porous microparticles.
U.S. Pat. No. 6,794,370, issued to Achterrath in 2004, discloses a method for treating metastatic colorectal cancer using CPT-11, 5-fluorouracil, and folinic acid as syngergistic combination therapy.

However, none of the references and patents mentioned above, taken individually or in any combination, describes the present invention as claimed.

Accordingly, there is a need in the field to invent such a product. As can be seen from the data discussed below, this novel polysaccharide-based 5-FU prodrug possesses enhanced target specificity to colorectal cancer cells. This unique property of the invention can lead to a higher efficacy and/or a reduced toxicity profile, thus providing a preferential method to deliver 5-FU for colorectal cancer treatment.

### SUMMARY OF THE INVENTION

The invention is directed to prodrug wherein 5-FU is coupled to polysaccharides containing galactose through different bridge linkage to obtain therapeutic conjugates (Huang et al, 2002; Li et al, 2003). Because of this specific linkage, the 5-FU prodrug cannot be digested or hydrolyzed in the upper gastrointestinal tract, and therefore is delivered specifically to the colorectal area. Once arriving at the colorectal area, the 5-FU will be released from the galactose-containing polysaccharide by hydrolysis of the prodrug by bacterial enzymes in the lower intestinal tract to release 5-FU-galactose, which binds galectin-3, a protein highly expressed in colorectal cancer (Schoeppner et al, 1995, Yoshii et al, 2002) to achieve a targeting action at the colorectal cancer cells.

Therefore, using large molecules of polysaccharide containing galactose as the carrier of 5-FU will not only achieve local release of drug in the colorectal area, but also have the targeting effect specifically at the colorectal cancer cells, resulting in enhanced therapeutic effects of 5-FU. With increased selectivity, improved safety profile will result, allowing the flexibility for oncologist to either optimize the dosage for maximal therapeutic effects or reduction in dosage for the elderly or frail patients. Moreover, many polysaccharides also have immunoregulatory function in addition to anti-tumor effects. This may be able to help reduce the immunosuppression effect from 5-FU. This invention therefore combines the medical design concepts of drug delivery, targeting, and synergism to achieve the goal of high efficacy and low toxicity.

### BRIEF DESCRIPTION OF THE DRAWING

Other objects, features, and advantages will be apparent from the following detailed descriptions of preferred embodiments taken in conjunction with the accompanying drawings in which:

FIG. 1. For illustration purposes only, an embodiment of the inventive drug delivery system wherein the galactose-containing polysaccharide is pectin, and Z refers to 5-FU. The symbols “**” and “*” indicate the position of β(1-4) glycosidic linkages, and n is from 1 to about 25,000.

### DETAILED DESCRIPTION OF INVENTIVE EMBODIMENTS

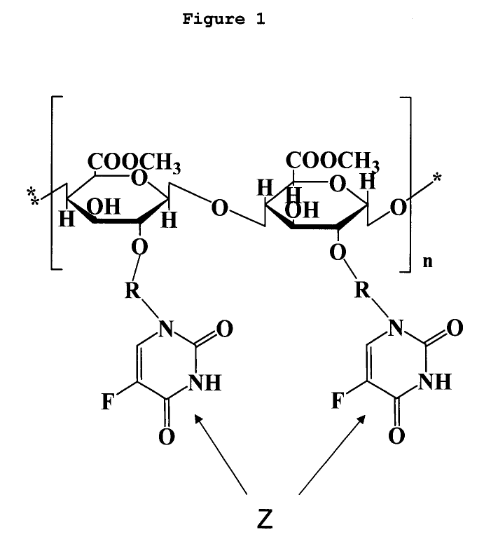

The purpose of this invention is to provide a novel prodrug and methods of its preparation, for the targeted treatment of colorectal cancer as illustrated in FIG. 1 (5-FU prodrug). First, 5-FU prodrug directs 5-FU to the colorectal area, and then the 5-FU-galactose is bound to galectin-3, which is a highly expressed protein in colorectal cancer cells to achieve specific release of the drug for the targeted treatment of colorectal cancer (Schoeppner et al, 1995, Yoshii et al, 2002). This design can increase 5-FU selectivity, enhance its therapeutic effects, and reduce systemic toxicities.

### DEFINITIONS

The following terms are used as defined herein. The use of these terms does not preclude the use of other terms not defined herein that are essentially synonymous with the defined terms.

The term prodrug refers to a compound whose efficacy is greatly enhanced after one or more conversion step(s) that occurs in vivo after administering the compound to a subject or patient.

The term galactose-containing polysaccharide refers to a polysaccharide having at least one galactose residue. A galactose-containing polysaccharide may be naturally occurring or may be prepared by modifying a different polysaccharide. Further, a galactose-containing polysaccharide may comprise unmodified galactose residues or modified galactose-derived residues.

The term galactose-containing fragment refers to a portion of the galactose-containing polysaccharide that may arise from being acted on by various enzymes. Enzymes that will generate galactose-containing fragments are largely expected in the colon. These enzymes are largely bacterial in nature.

The term therapeutic parent compound refers to a compound having therapeutic and/or diagnostic properties in a form prior to its linkage to a galactose-containing polysaccharide. The term therapeutic parent compound is interchangeable with either parent compound or parent therapeutic compound.

The term derivatize or derivatizing refers to modifying a compound, e.g., galactose-containing polysaccharide or a therapeutic parent compound, by adding one or more reactive groups to the compound by reacting the compound with a functional group-adding reagent. As used herein, the term also refers to the attachment of cross-linkers to the compounds. The cross-linkers may be bifunctional, thus reacting with both compounds. A cross-linker possesses spacer arms that vary in size in different cross-linking compounds. This may be useful if one elects to have a known fixed distance between the galactose-containing polysaccharide and therapeutic parent compound.

The term linkage or linking bond, refers to the covalent bond connecting, or linking, the galactose-containing polysaccharide and the therapeutic parent compound. This bond may be formed by attaching one or more functional groups to either of, or both of, the therapeutic parent compound and galactose-containing polysaccharide. The galactose-containing polysaccharide and/or the therapeutic parent compound may be derivatized by addition of the functional groups.

The term conjugate as used herein refers to the prodrug of the structural formula galactose-containing polysaccharide-R-Z.

The embodiments described encompass a prodrug for targeted treatment of colorectal cancer. Further embodiments include the methods of preparing the prodrug, and its use in vivo as an illustrative example. The embodiment in FIG. 1 is for the treatment of the colorectal cancer and comprises a linkage of polysaccharides with 5-FU through various bridging covalent linkages. The prodrug has the following features:
The polysaccharides are galactose-containing polysaccharides.
The galactose-containing polysaccharides may be purified from natural gums and plants.
The natural gums are pectin, guar gum, and carob bean gum, and the plant polysaccharides include aloe polysaccharide, medlar polysaccharide, and rhubarb polysaccharide.
The prodrug's structure may be represented as Polysaccharide-R-Z, in which R comprises one of the following functional groups: —(CH₂)ₙ—, —CO—, —CO(CH₂)ₙ—, and —CO(CH₂)ₙ—CO—, —NH(CH₂)ₙ—, —CONH—, —CONR₂—, —O—NH—, —NH(CH₂)ₙ—, —CONH—, —CONR₂—, —O—NH wherein n is from 1 to 5, and R₂ is a branched, unbranched or cyclic, aliphatic or aromatic having from 2 to 20 carbon atoms.
The specific embodiment of the prodrug wherein Z is 5-FU would have the structure Polysaccharide-R-5-FU, in which R comprises one of the following functional groups: —(CH₂)ₙ—, —CO—, —CO(CH₂)ₙ—, and —CO(CH₂)ₙ—CO—, —NH(CH₂)ₙ—, —CONH—, —CONR₂—, —O—NH—, —NH(CH₂)ₙ—, —CONH—, —CONR₂—, —O—NH—, wherein n is from 1 to 5, and R₂ is a branched, unbranched or cyclic, aliphatic or aromatic having from 2 to 20 carbon atoms
The pectin, guar gum, and carob bean gum are hydrolyzed first with alkali (pH=9-10) then with acid (pH=3-5), and precipitated in alcohol and dialyzed to produce natural gums of target molecular weights of approximately 1 to approximately 10⁷ Da.

The extraction method for the aloe polysaccharide, medlar polysaccharide, and rhubarb polysaccharide is as follows: First, pulverized the aloe/medlar/rhubarb plant material, and boiled with ethanol for three eight-hour-periods. The components dissolved in ethanol are extracted. The residue is boiled with water for another three eight-hour-periods in order to extract polysaccharides. All the water extractions are then collected. The polysaccharide-enriched fractions are obtained by precipitation with 5 volumes of ethanol for 3 times. After removing proteins dialysis, separate and purify with gel filtration chromatography, the polysaccharide components are obtained with molecular weights of 10⁵-10⁷ Da.

During the extraction process, the following analytical instrumentation and techniques are implemented: a). High-performance liquid chromatography (HPLC) for purity analysis; b). Ultraviolet (UV) and infrared spectroscopic identification for qualitative examination; c). Measurement of sugar and glycuronic acid contents respectively by vitriol-phenol and vitriol-carbazole methods; and d). Measurement of the monosaccharide compositions of the polysaccharides of different molecular weights and their component ratio was performed by chromatographic techniques and gas chromatography.

An illustrative embodiment of a method for preparing the prodrug is exemplified by use of 5-FU as the parent therapeutic compound. Specifically, a method of attaching 5-FU to a galactose-containing polysaccharide may embody forming the prodrug by:
1. forming a covalent attachment between the 5-FU and a free hydroxyl group in the galactose-containing polysaccharide via the formation of an ester or ether linkage;
2. forming a covalent attachment between the —NH of 5-FU at the free hydroxyl group of the galactose residues contained in the polysaccharides via the formation of an acylamide or acylamine linkage.

Generally, the synthetic method for the prodrug is to bond with the parent compound at the free hydroxyl group of the galactose residues contained in the polysaccharide via the formation of an ester or ether linkage through modifying the free hydroxyl group to, e.g., a reactive carboxyl group (e.g. a carboxylic acid chloride). Then, the —NH₂ or similar functional groups (including —NHNH₂, —ONH₂, —NHC═(O)NHNH₂, —OH, —CO₂H, and —SH) of the therapeutic parent compound reacts the modified carboxyl group of the galactose residues contained in the polysaccharides via the formation of an ester, ether, amine, amide, acyl amine, a thioether or a thioester.

The formation of the ester linkage can be made, for example, through the acyl chloride method or N,N′-dicyclohexylcarbodiimide (DCC) methods. The formation of the ether linkage may be carried out through condensation. The formation of the acyl-amine linkage is derived from aminolysis of acyl chloride.

For illustration purposes an embodiment of the inventive drug delivery system is described below.

Pectin: a polysaccharide composed of straight chains of galacturonic acid. The symbols “**” and “*” indicate the position of β(1-4) glycosidic linkages, and n is from 1 to about 12,500.

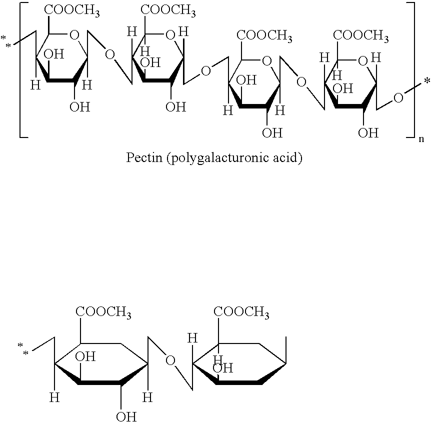

Guar gum: a non-ionic polysaccharide mainly polymerized with galactose and mannose, belonging to natural galactomannan with mannose as its main chain and β(1-4) glycoside link as the linkage between D-mannopyranose units. Meanwhile, galactopyranose is connected to the mannose main chain through α(1-6) link. The molar ratio between mannose and galactose is 2:1.

Carob bean gum: a colorless and flavorless polysaccharide refined from plant endosperm, mainly containing mannose and galactose with an average molecular weight of 300 kDa.

It is currently known that the natural occurring gums containing galactose residues, such as pectin, guar gum, and carob bean gum have the functions of regulating the bacterial colonies in the intestinal tract as well lowering cholesterol. In addition, aloe polysaccharides, medlar polysaccharides, and rhubarb polysaccharides are rich in galactose with known immunoregulation functions, which have not, as of yet, been fully explored for pharmaceutical development.

Preparation Method of the Novel Prodrug for the Targeted Treatment of Colorectal Cancer

The preparation methods of the prodrug involved in this invention and its characteristic of release in the colorectal area will be described below. The new uses of the inventive 5-FU prodrug in drug delivery and therapeutic use will also be dealt with through the results of pharmacological testing in a mouse model where the mice have colorectal cancer induced by 1,2-dimethylhydrazine (DMH) and Dextran Sulfate Sodium (DSS). However, this invention is not limited to the examples described below.

The natural gums containing galactose residues are hydrolyzed first with alkali (pH=9-10) then with acid (pH=3-5), and precipitated with alcohol and dialyzed to obtain natural gums of targeted molecular weights (10⁵˜10⁷ Da) containing galactose residues.

The extraction method for galactose such as aloe polysaccharide, medlar polysaccharide, and rhubarb polysaccharides is to pulverize the aloe/medlar/rhubarb plant materials, and boil with ethanol for three eight-hour-periods. The components dissolved in ethanol are then extracted. The residue is boiled with water for another three eight-hour-periods in order to extract polysaccharides. All the water extractions are then collected. The polysaccharide-enriched fractions are obtained by precipitation with 5 volumes of ethanol for 3 times. After removing proteins, dialysis, separate and purify with gel filtration chromatography, the polysaccharide components are obtained with molecular weights of approximately 10⁵˜10⁷ Da. During the extraction process, HPLC for purity analysis, UV and infrared spectroscopic identification for qualitative examination, measurement of sugar and glycuronic acid contents respectively by vitriol-phenol and vitriol-carbazole methods, and measurement of monose compositions of the polysaccharides of different molecular weights and their component ratio with chromatographic techniques and gas chromatography are performed.

Link the above-mentioned polysaccharides (including those prepared from natural gums) with 5-FU, and the linkage method can be acetylating the aforementioned polysaccharides first, and then connecting them with 5-FU under different conditions as per Example 1; and can also be acetylating 5-FU first, and then connect it with the aforementioned polysaccharides under different conditions as per Examples 2 and 3.

This method includes connecting 5-FU with the hydroxyl group of polysaccharides through derivation to form an ester or ether linkage, or chemically linking polysaccharides with the —NH part of the 5-FU to form an acylamine linkage through derivation. The formation of the ester linkage is carried out through acyl chloride method or N,N′-dicyclohexylcarbodiimide (DCC) method. The forming of the ether linkage is carried out through condensation, and the formation of the acyl-amine linkage is derived from aminolysis of acyl chloride.

### ILLUSTRATIVE EMBODIMENTS

In view of the foregoing disclosure several embodiments of the prodrug and its methods of preparation are apparent. The following embodiments are presented for illustrative purposes only and are not meant to limit the scope of the claimed subject matter. Persons of ordinary skill in the art may be able to describe further embodiments based on the guidance set forth in the foregoing disclosure, the examples below and knowledge in the art.

A desirable embodiment is an anti-cancer prodrug with target specificity toward colorectal cancers. The prodrug is synthesized by chemically linking a uniquely prepared polysaccharide with 5-FU through one or more bridge links. The molar ratio of 5-FU to the galactose-containing polysaccharide may thus be more than 1:1.

Additional embodiments are illustrated by a prodrug for the targeted treatment of colorectal cancers and the prodrug's method of preparation. For example, the polysaccharide used in preparing the prodrug contains galactose residues.

It is also desirable to provide embodiments of the prodrug for the targeted treatment of colorectal cancers wherein the galactose-containing polysaccharide is prepared from naturally occurring gums or plant material. These embodiments of the prodrug may have a galactose-containing polysaccharide prepared from pectin, guar gum, and carob bean gum, and the plant materials aloe, medlar and rhubarb. However, virtually any plant material having galactose-containing polysaccharides would make a suitable starting material for isolating said galactose-containing polysaccharide.

The embodiments of the prodrug for targeted treatment of colorectal cancers may have the parent compound 5-FU directly or indirectly linked to a galactose-containing polysaccharide. An indirect linkage is defined as a linkage between 5-FU and a galactose-containing polysaccharide that is mediated by a bifunctional crosslinker of the kind that are commercially available. Direct linkages, in contrast, do not employ a crosslinking agent in linking 5-FU and galactose-containing polysaccharides. Instead an unmodified or derivatized galactose-containing polysaccharide is bonded directly to one or more 5-FU molecules.

In additional embodiments, it is the parent compound 5-FU that is derivatized and linked to an unmodified or derivatized galactose-containing polysaccharide. It is understood to those in the art that derivatizing, as used herein, refers to the addition of reactive functional groups to a galactose-containing polysaccharide or a 5-FU molecule without introducing the spacer arms that characterize the addition of commercially available cross-linkers. However, it is also readily appreciated by persons of ordinary skill in the art that whether the conjugated prodrug is formed by directly linking 5-FU to a galactose-containing polysaccharide, or by using crosslinkers, equally effective prodrug conjugates can be obtained. In part, the effectiveness of the prodrug conjugate is based on its having significant colorectal-targeting specificity to deliver the 5-FU to cancer cells.

Whether the linking is direct or indirect, the prodrug possesses the structural formula Polysaccharides-R-5-FU, in which the R is a group that provides the linkage between a galactose-containing polysaccharide and 5-FU and where R can comprise any of the following functional groups: —(CH₂)—, —CO—, —CO(CH₂)ₙ—, —CO(CH₂)ₙCO—, and n=1, 2, 3, or 4.

An embodiment of the prodrug may result from forming a covalent linkage between the 5-FU and free hydroxyl groups of the galactose residues in the polysaccharides. This linkage may be achieved via the formation of ester or ether linkages through derivatization. An illustrative example would be to form the bond between the —NH of 5-FU and free hydroxyl groups of galactose residues in the polysaccharides via the formation of an acylamide or an acylamine linkage through derivation. Embodiments of the methods for preparing the prodrug may also use as starting material for galactose-containing polysaccharide isolation, pectin, guar gum, or carob bean gum. Either material is first hydrolyzed with alkali (pH=9-10) then with acid (pH=3-5), and followed by precipitation with alcohol and dialysis. These methods yield galactose-containing polysaccharides of molecular weights from approximately 10⁵ Da to approximately 10⁷ Da.

Additional embodiments of the methods for preparing the prodrug may comprise isolating the galactose-containing polysaccharides from aloe, medlar or rhubarb as follows: pulverizing the aloe/medlar/rhubarb plant material, and boiling with ethanol for three eight-hour-periods. The components dissolved in ethanol are extracted. The ethanol insoluble residue is boiled with water for another three eight-hour-periods in order to extract polysaccharides. All the water extractions are then collected. The polysaccharide-enriched fractions are obtained by precipitation with 5 volumes of ethanol for 3 times. After removing proteins, dialysis, separate and purify with gel filtration chromatography, the polysaccharide components are obtained with molecular weights of about 10⁵˜10⁷ Da. During the extraction process, high-performance liquid chromatography (HPLC) for purity analysis, ultraviolet (UV) and infrared spectroscopic identification for qualitative examination, measurement of sugar and glycuronic acid contents respectively by vitriol-phenol and vitriol-carbazole methods, and measurement of monosaccharide compositions of the polysaccharides of different molecular weights (weight-average molecular weight) and their component ratio with chromatographic techniques and gas chromatography are performed.

An embodiment of the linking methods for linking a galactose-containing polysaccharide and 5-FU is to that the formation of the ester linkage is made through acyl chloride method or N,N′-dicyclohexylcarbodiimide (DCC) method, the forming of the ether linkage is carried out through condensation, and the formation of the acyl-amine linkage is derived from aminolysis of acyl chloride.

The embodiments of the prodrug illustrated above are effective for the treatment of colorectal cancers. Further, it is known that colorectal cancer cells express a galactose-binding lectin, galectin-3. Galectin-3 has also been localized in cancers of the breast, lung, prostate, bladder, thyroid, lymphoma, pancreas, gastrointestinal and head and neck cancers. Accordingly, the embodiments of the prodrug and its method of preparation that are encompassed by the specification may have utility in treating a broad spectrum of cancers in additional to colorectal.

The examples described below provide illustrative embodiments of methods of preparing the inventive prodrug. It should be readily appreciated that these examples taken together with knowledge in the art would allow persons in the art to practice related_embodiments that are clearly encompassed by the subject matter disclosed and claimed herein.

### EXAMPLES

### Example 1

Add 1.2 g of pectin into 52.5 g (0.56 mmol) of melting chloroacetic acid and stir it in solution under 70° C. constant temperature, and then add 35 ml of acetic anhydride. Stir it for 3 hr at a constant temperature of 70° C., pour the solution into a large amount of ice water, forming a yellow precipitate. Separate out the yellow gel-like precipitate, wash it thoroughly with water and ethanol respectively in sequence, collect the precipitate by filtration, and dry it under vacuum at 40° C. for 24 hr to obtain a grayish yellow powder of chloroacetyl pectin.

Weigh 0.38 g of this chloroacetyl pectin and add into 20 ml of dimethyl sulfoxide (DMSO), stir it under 60° C. until it is dissolved. Then put a mixture of 0.65 g of 5-FU and triethylamine into the above-mentioned solution, stir it for 24 hr under 60° C. constant temperature, and then pour the solution into 100 ml of anhydrous mixture ethanol-ether (1:1 ratio) to produce a loose fluffy precipitation. Let it stand still thoroughly, filter it by vacuum, wash it thoroughly with anhydrous ethanol, and dry it under vacuum at 40° C. for 24 hr to obtain a light yellow precipitate of pectin-5-FU.

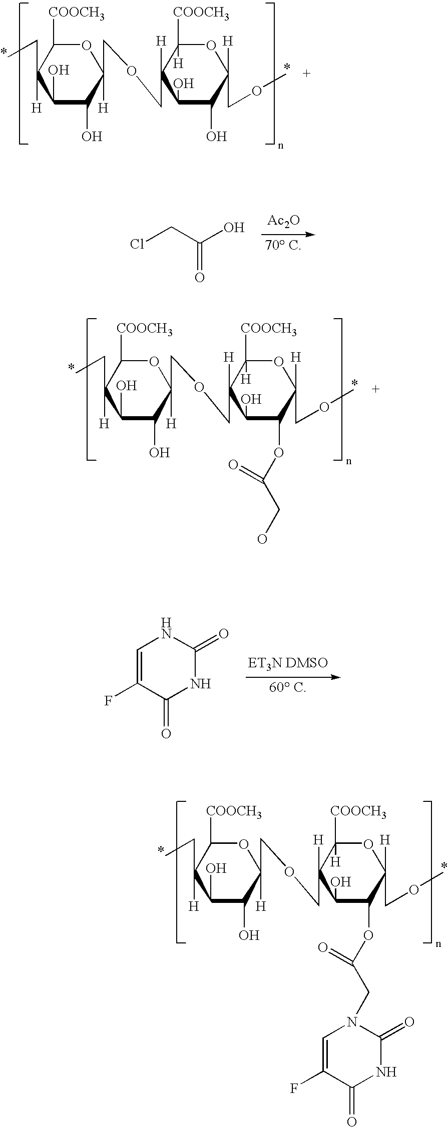

### Example 2

Dissolve 3.92 g of 5-FU and 3.65 g of sodium hydroxide (NaOH) in 22 ml of water, add 12 ml of aqueous solution of 3.30 g of chloroacetic acid, maintain at pH 10, reflux for 2 hr, and acidify the solution using concentrated HCl to obtain a light brown precipitate. Recrystallize it to obtain 2.26 g of white solid with a yield of ˜40%.

Dissolve 0.5 g of carob bean gum in 20 ml of DMSO, add 0.25 g of N,N′-dicyclohexylcarbodiimide (DCC) and 15 mg of 4-dimethylaminopyridine (DMAP), and then add 0.5 g of 5-Fu-1-acetic acid, stirring for 24 hr at 40° C. At completion, pour the reaction mixture into ethanol forming a jelly-like substance. Filter off the jelly-like substance, rinse it with methanol, and then dry under vacuum to obtain final product.

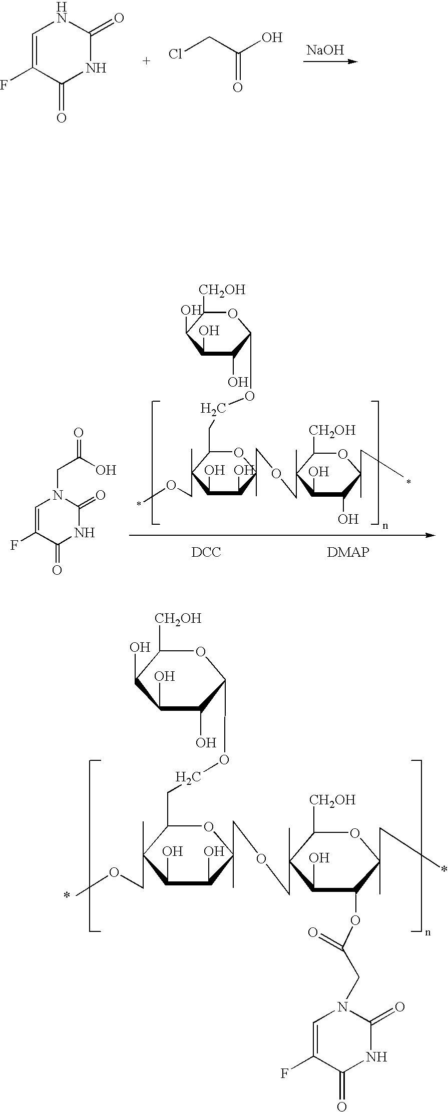

### Example 3

Add 1.0 g of 5-FU in 20 ml of pyridine and stir thoroughly to dissolve the contents into solution. Cool it down to 0° C. in an ice water bath. Add 2 ml of trichloromethyl chloroformate (TCF) slowly dropwise into this 5-FU pyridine solution over 30 minutes. Stir reaction continuously for 1 hr. Remove reaction mixture from the ice water bath. While continuously stirring, allow reaction mixture to warm to room temperature over 2 hr, and then heat reaction mixture to 40° C. and let reaction continue for 30 minutes. Reduce the pressure to remove the unreacted phosgene and pyridine to obtain the brown solid product of chloroformyl 5-FU. Rinse the product with tetrahydrofuran (THF), filter it by vacuum, and dry it by vacuum drying for 6 hr.

Weigh 1 g of guar gum and dissolve it in 20 ml of DMSO. Add 5 ml of pyridine, stir, and heat the mixture to 40° C. Allow the contents to be dissolved thoroughly, add the chloroformyl 5-FU, stir continuously at room temperature for 24 hr, then heat to 40° C. and allow reaction to continue for 16 hr. The product is precipitated by adding excess anhydrous ethanol-ether (1:1 ratio) and filtered under vacuum. The precipitate is then re-dissolved in DMSO, precipitated with anhydrous ethanol-ether (1:1 ratio), and vacuum filtered. Repeat for two more times to obtain a brown solid product pectin-CH₂—CO-5-FU and then dry under vacuum for 24 hr.

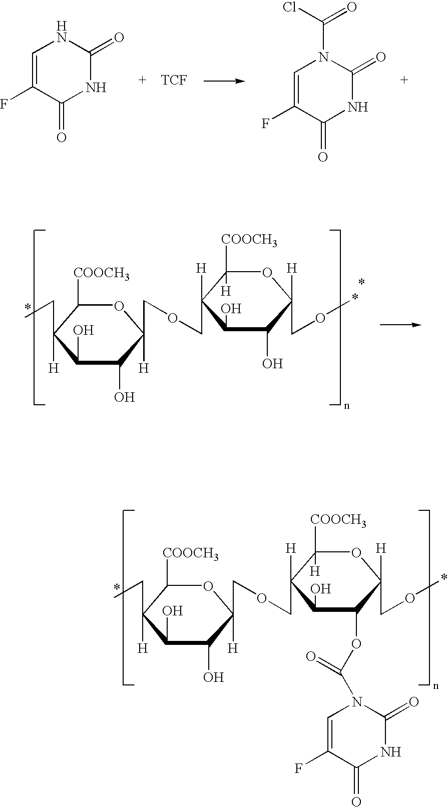

### Example 4

Mix up 5 g of malonic acid, 3 g of benzyl alcohol, 20 ml of toluene, and 100 mg of p-toluene-sulphonic acid (TsOH), and stir it thoroughly, heating to 120° C., and reflux it with a water separator for 1 hr to remove the water. Dissolve the residue in ethyl acetate (60 ml), and then wash with saturated NaCl brine (15 ml) three times. Using a separatory funnel, extract the reaction product from the organic layer (ethyl acetate) first with 1 M NaOH (30 ml), then with saturated NaHCO₃ (10 ml), saving both aqueous layers. Repeat this extraction sequence again, saving both aqueous layers. After combining the aqueous layers, add 20 ml of chloroform, and add concentrated hydrochloric acid slowly dropwise while stirring until the water layer is not turbid. Separate and save the organic layer (chloroform), and then extract from the aqueous layer with 10 ml of chloroform twice, saving the organic layers. Combine the extracted chloroform layers and wash them with 10 ml of water twice. Dry them using anhydrous sodium sulfate. Distill off the chloroform by vacuum evaporation and concentrate them to obtain 2.3 g of an oily yellow substance of benzyl malonic acid (compound 1).

Add 0.78 g of 5-FU into 5 ml of hexamethyl aminosilane and heat to 145° C. Add trimethyl chlorosilane slowly dropwise, stir and reflux it for 4 hr, and then distill off the excess hexamethyl aminosilane to obtain the clear, colorless crystal of 2,4-bis(trimethylsilaneoxy)-5-FU (compound 2) for the next reaction step.

Add 2.3 g of compound (I) into 8 ml of thionyl chloride. After stirring and refluxing it for 3 hr at 60° C., distill off the excess thionyl chloride by vacuum evaporation to obtain 2-benzyloxycarbonyl-acetyl chloride. Dissolve this compound into 8 ml of anhydrous acetonitrile, then add it to the above-mentioned 2,4-bis(trimethylsilaneoxy)-5-FU under nitrogen atmosphere. Add 1.68 ml of triethylamine, and reflux for 4 hr at 75° C. Distill off the solvent by vacuum evaporation to obtain a pale brown solid. Recrystallize it in toluene twice to obtain 0.7 g of a white solid, which is 3-(5′-fluoro-3′H-2′4′-pyridine diketone)-3-oxo-benzyl propionate (compound 3).

Dissolve 0.7 g of compound (3) in anhydrous tetrahydrofuran (THF), and add 10% palladium-carbon mixture (Pd—C), stir at room temperature and bubble with hydrogen gas at atmospheric pressure for 24 hr. Filter the reaction product and concentrate it by vacuum evaporation to obtain 0.5 g of a white solid, which is 3-(5′-fluoro-3′H-2′4′-pyridine diketone)-3-oxo propionic acid (compound 4).

Weigh 0.5 g of the prepared aloe polysaccharide and dissolve in 20 ml of DMSO, add 0.25 g of N,N′-dicyclohexylcarbodiimide (DCC) and 15 mg of 4-dimethylaminopyridine (DMAP), and 2.5 g of compound (4). Stir the contents at 35° C. for 48 hr. Pour the reaction mixture into ethanol forming a jelly-like substance. Filter the jelly-like substance, rinse it with methanol, and then dry under vacuum to obtain final product.

The above linkage method can be substituted using other dicarboxylic acids, where the malonic acid (—COCH₂CO) is substituted with succinic acid —CO(CH₂)₂CO—, glutaric-CO(CH₂)₃CO—, or adipic acid —CO(CH₂)₄CO—, etc.

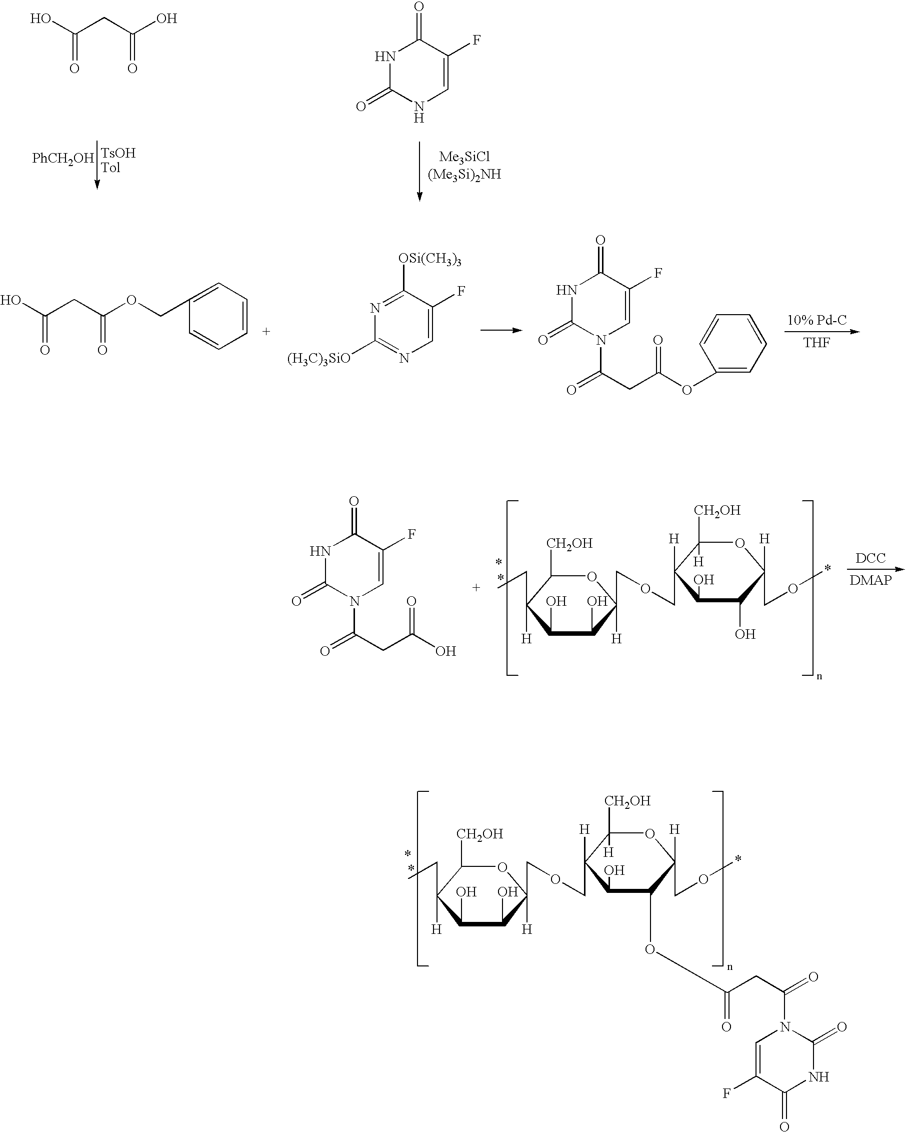

### Example 5

Perform the same initial procedures as described in Example 4 up to the production of 3-(5′-fluoro-3′H-2′4′-pyridine diketone)-3-oxo propionic acid (compound 4).

Weigh 0.6 g of the prepared medlar polysaccharide and dissolve into 20 ml of DMSO, add 0.25 g of DCC, 15 mg of DMAP, and 2.5 g of compound (4), stirring at 35° C. for 48 hr. Pour the reaction mixture into ethanol forming a jelly-like substance. Filter the jelly-like substance, rinse it with methanol, and then dry under vacuum to obtain final product.

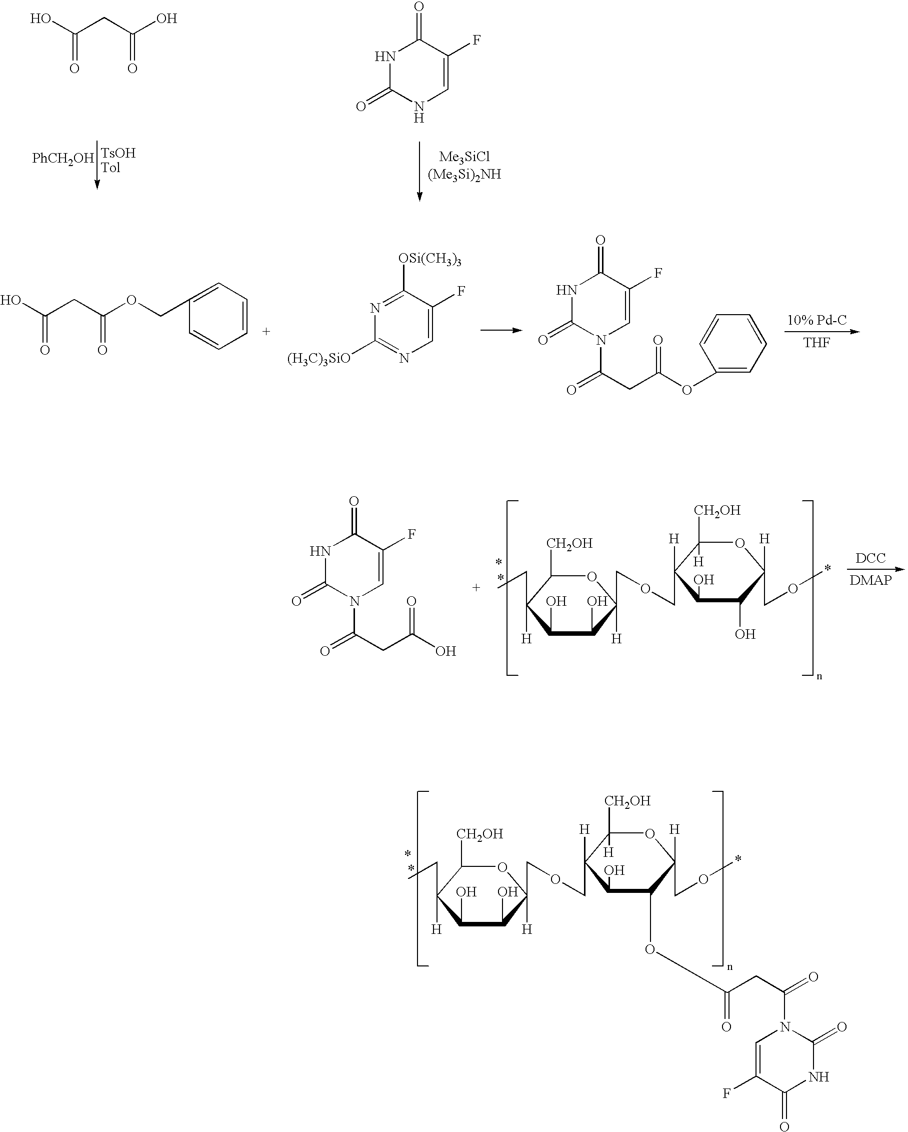

The above linkage method can be substituted using other dicarboxylic acids, where the malonic acid (—COCH₂CO) is substituted with succinic acid —CO(CH₂)₂CO—, glutaric-CO(CH₂)₃CO—, or adipic acid —CO(CH₂)₄CO—, etc.

### Example 6

Perform the same initial procedures as described in Example 4 up to the production of 3-(5′-fluoro-3′H-2′4′-pyridine diketone)-3-oxo propionic acid (compound 4).

Weigh 0.90 g of rhubarb polysaccharide and dissolve in 20 ml of DMSO, add 0.25 g of DCC, 15 mg of DMAP, and 2.5 g of compound (4), stirring at 35° C. for 48 hr. Pour the reaction mixture into ethanol forming a jelly-like substance. Filter the jelly-like substance, rinse it with methanol, and then dry under vacuum to obtain final product.

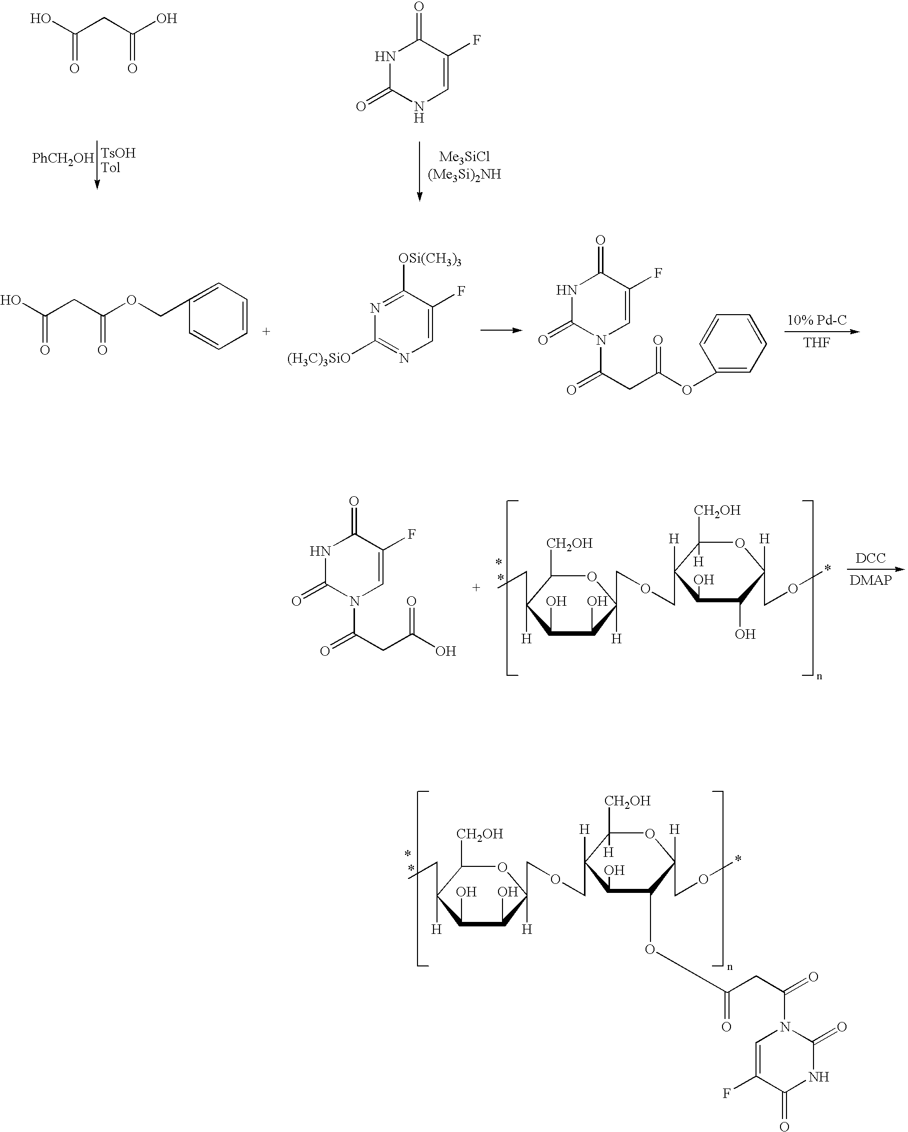

The above linkage method can be substituted using other dicarboxylic acids, where the malonic acid (—COCH₂CO) is substituted with succinic acid —CO(CH₂)₂CO—, glutaric-CO(CH₂)₃CO—, or adipic acid —CO(CH₂)₄CO—, etc.

This invention is not limited to the examples as described in these specifications. The examples are for illustration only. The actual pharmaceutical forms of this invention can be tablet, capsule, geltab, caplet, liquid, suspension, drops, gel, syrup, slurry, tincture, lozenge, gum, or any other suitable oral formulation in any appropriate vehicle to be used for cancer patients.

### Example 7

### Pharmacokinetic Characteristics of this Novel 5-FU Prodrug

Regular generic 5-FU when fed orally to mice is mainly distributed to the mucosa of stomach and proximal part of the small intestine, and much less to the distal small intestine (ileum, cecum) or colon and rectum. On the other hand, when the current 5-FU prodrug is fed to mice orally, free 5-FU cannot be detected in the mucous membrane of the stomach or proximal small intestine. Instead, the majority of this novel drug is detected in the distal ileum, cecum, colon, and rectum, illustrating that this novel 5-FU prodrug is mainly released in these areas for its targeting function. Table 1 and 2 depict experiments that illustrate these findings.

Note that the dashed lines represent undetectable levels of 5-FU.

TABLE 1
Distribution of 5-FU in Different Parts of the GI Tract after Regular Generic 5-FU
(22.5 mg/kg) is Orally Fed to the Tested Mice (n = 6, x̅ ± s)

| | 5-FU (μg/g) | | | | |
|---|---|---|---|---|---|
| | | Proximal | Distal | | |
| Time | | Small | Small | Ileum and | Colon and |
| (hr) | Stomach | Intestine | Intestine | Cecum | Rectum |
| 1 | 3.04 ± 0.49 | 1.75 ± 0.24 | 0.42 ± 0.21 | — | — |
| 2.5 | 1.97 ± 0.31 | 0.64 ± 0.42 | 0.43 ± 0.24 | 0.37 ± 0.12 | 0.25 ± 0.12 |
| 3.5 | 1.12 ± 0.46 | 0.53 ± 0.32 | 0.62 ± 0.12 | 0.42 ± 0.21 | 0.41 ± 0.24 |
| 4.5 | 0.97 ± 0.23 | 0.42 ± 0.91 | 0.35 ± 0.21 | 0.37 ± 0.12 | 0.56 ± 0.06 |
| 6 | 0.63 ± 0.31 | 0.37 ± 0.05 | 0.31 ± 0.09 | 0.21 ± 0.11 | 0.17 ± 0.04 |
| 9 | 0.33 ± 0.18 | 0.17 ± 0.04 | 0.17 ± 0.06 | 0.03 ± 0.01 | 0.08 ± 0.05 |
| 12 | 0.06 ± 0.03 | 0.03 ± 0.01 | 0.07 ± 0.04 | 0.04 ± 0.02 | 0.05 ± 0.02 |
| 15 | — | — | — | — | — |

TABLE 2
Distribution of 5-FU in Different Parts of GI Tract after
the Novel 5-FU Prodrug (22.5 mg/kg) is Orally Fed to the
Experimental Mice (n = 6, x̅ ± s)

| | 5-FU (μg/g) | | | | |
|---|---|---|---|---|---|
| | | Proximal | Distal | | |
| Time | | Small | Small | Ileum and | Colon and |
| (hr) | Stomach | Intestine | Intestine | Cecum | Rectum |
| 1 | — | — | — | — | — |
| 2.5 | — | — | — | — | — |
| 3.5 | — | — | — | — | — |
| 4.5 | — | — | — | — | — |
| 6 | — | — | — | 0.32 ± 0.18 | 0.18 ± 0.02 |
| 9 | — | — | — | 0.32 ± 0.10 | 0.57 ± 0.21 |
| 12 | — | — | — | 0.11 ± 0.05 | 0.13 ± 0.09 |
| 15 | — | — | — | — | — |

### Example 8

### Toxicological Effects of 5-FU Versus 5-FU Prodrug

Normal mice are administered with 7.5, 22.5, or 67.5 mg/kg/d of 5-FU and the 5-FU prodrug (as equivalent 5-FU dose). There are no obvious toxic effects in the first dose for both treatments. However, when both drugs are administered for seven consecutive days, the toxicity profile of the 5-FU prodrug is significantly better than that of 5-FU. Table 3 illustrates this observation:

TABLE 3
The Survival Rate of Normal Mice Administered
with 5-FU and 5-FU Prodrug (n = 10)
Dosage (mg/kg/d)
Survival Rate (%) after 7-day Treatment

| | |
|---|---|
| 5-FU | 5-FU Prodrug |

| | | | | | |
|---|---|---|---|---|---|
| 7.5 | mg/kg/d | 100% | (10/10) | 100% | (10/10) |
| 22.5 | mg/kg/d | 70% | (7/10) | 100% | (10/10) |
| 67.5 | mg/kg/d | 0% | (0/10) | 80% | (8/10) |

### Example 9

### Therapeutic Effects of the Current 5-FU Prodrug on Colorectal Cancer

5-FU has a great efficacy in an animal model of mice possessing colorectal cancer induced by 1,2-dimethylhydrazine (DMH) and Dextran Sulfate Sodium (DSS). However, 5-FU also possesses the well-known hematological toxicity of bone marrow suppression. On the other hand, when the current novel 5-FU prodrug is used in the same animal model, its therapeutic effect is greatly enhanced. In addition, the dosage of 5-FU needed can be reduced such that the toxicity caused by 5-FU can be minimized as well. Table 4 depicts the therapeutic effect when this 5-FU prodrug and a generic 5-FU are compared to treat mice with induced colorectal cancer. The doses delivered are equivalent with 1 time per day for 7 days as one treatment period, and then another period of treatment after 7 days of rest (i.e., a total of 21 days, two treatment periods with one week of rest in between).

It can be seen from the results shown in Table 4 below that the 5-FU prodrug can lead to an increased survival rate (from either lower drug toxicities or slower cancer progression) in the treated mice as well as a decrease in tumor weight. When the dose of the 5-FU prodrug is just 33% of the generic 5-FU, the therapeutic effects are essentially identical. On the other hand, when the dosages of the two drugs are both at 22.5 mg/kg/d, all mice treated with 5-FU prodrug survived compared to only half (100% vs. 50%) for the generic 5-FU treated mice. In addition, the tumor burden (expressed as tumor weight) is also much less (19% vs. 65%) showing that the therapeutic effect of this 5-FU prodrug on colorectal cancer is obviously better than that of generic unmodified 5-FU.

TABLE 4
Therapeutic Effects of 5-FU Prodrug on Mice with Colorectal Cancer
Induced by DMH-DSS (n = 10 per group, x̅ ± s)

| | Dosage of | | | | Tumor Weight |
|---|---|---|---|---|---|
| | Equivalent 5-FU | Survival at | | | as Compared |
| | Administered | the End of | Survival | Tumor Weight | to the Saline |
| Group | (mg/kg/d) | the Study | Rate | Index (mg/g) | Control |
| Normal Mice Control | 0 | 10/10 | 100% | — | — |
| (No Cancer treatment) | | | | | |
| Control (Saline) | 0 | 5/10 | 50% | 689 ± 126.7 | 100% |
| Generic | 22.5 | 5/10 | 50% | 447 ± 113.5 | 65% |
| 5-Fluorouracil | | | | | |
| (5-FU) | | | | | |
| Polysaccharide-Linked | 7.5 | 7/10 | 70% | 469 ± 124.9 | 68% |
| 5-Fluorouracil | | | | | |
| prodrug (P-5-FU) | | | | | |
| Polysaccharides Linked | 22.5 | 10/10 | 100% | 134.1 ± 67.4 | 19% |
| 5-Fluorouracil | | | | | |
| prodrug (P-5-FU) | | | | | |

However, it should be understood that the 5-FU prodrug exerts its efficacy after it is digested and hydrolyzed at the target sites in the body. Because it is a prodrug, it has no direct effect on in vitro colorectal cancer cell lines. For example, an in vitro test is conducted (details not enclosed) which showed the IC₅₀ of generic 5-FU for suppressing COLO-205 cell proliferation is 3.2×10⁻⁶ M, and the IC₅₀ for suppressing HT-29 cell proliferation is 13×10⁻⁶ M. However, as expected, there is no direct tumor suppression effect of this novel 5-FU prodrug on these two colorectal cancer cell lines shown.

Persons of ordinary skill in the art would readily appreciate that the foregoing descriptions of compounds and their methods of synthesis and use may be adopted for use with additional therapeutic compounds. Thus, the procedures described herein would provide sufficient guidance for preparing additional colon-targeted therapeutics of various types. Further, the methods and compounds described may be adopted as is, or modified according to need, for targeting therapeutic compounds to other galectin-3 expressing tumors or tissues.

This invention is not limited to the implementation examples as described in these specifications. The implementation examples are for illustration only. The actual pharmaceutical forms of this invention can be any suitable formulation to be used for patients.

### REFERENCES

Fan C L. Mai C S. Xu J J. Zhuo R X. Synthesis of Condensation Polymers of 1,3-Dihydroxylalkyl-5-Fluorouracil with Dichlorophsphates. Polymer Communications. 1(1):77-80, 1985. (Publication in Chinese)
Huang J. Gao C S. Mei X G. New Type of Colon Targeting Drug Delivery System. Foreign Medical Sciences Section on Pharmacy. 29(5):306-308 & 320, 2002. (Publication in Chinese)
Li G F. Chen J H. Liu L J. Guo D. Yan Y. Xu Z Y. Chen Z L. Absorption and Distribution of 5-Aminosalicylic Acid from its Chitosan Capsule Degraded by Colon Bacteria-Released Enzymes in Rats. Journal of First Military Medical School. 23(5):431-434, 2003. (Publication in Chinese)
Luo Y. Zhuo R X. Fan C L. Studies on the Synthesis and Antitumor Activity of Biodegradable Polyphosphamides. Chemical Journal of Chinese Universities. 15(5):767-770, 1994. (Publication in Chinese)
Ohya Y. Inosaka K. Ouchi T. Synthesis and antitumor activity of 6-O-carboxymethyl chitin fixing 5-fluorouracils through pentamethylene, monomethylene spacer groups via amide, ester bonds. Chemical & Pharmaceutical Bulletin. 40(2):559-61, 1992.
Ohya Y. Takei T. Kobayashi H. Ouchi T. Release behavior of 5-fluorouracil from chitosan-gel microspheres immobilizing 5-fluorouracil derivative coated with polysaccharides and their cell specific recognition. Journal of Microencapsulation. 10(1):1-9, 1993.
Ouchi T. Hagihara Y. Takahashi K. Takano Y. Igarashi I. Synthesis and antitumor activity of poly(ethylene glycol)s linked to 5-fluorouracil via a urethane or urea bond. Drug Design & Discovery. 9(1):93-105, 1992.
Schoeppner H L. Raz A. Ho S B. Bresalier R S. Expression of an endogenous galactose-binding lectin correlates with neoplastic progression in the colon. Cancer. 75(12):2818-26, 1995.
Yoshii T. Fukumori T. Honjo Y. Inohara H. Kim H R C. Raz A. Galectin-3 phosphorylation is required for its anti-apoptotic function and cell cycle arrest. Journal of Biological Chemistry. 277(9):6852-6857, 2002.
Zhu K J. Zhang J X. Wang C. Yasuda H. Ichimaru A. Yamamoto K. Preparation and in vitro release behaviour of 5-fluorouracil-loaded microspheres based on poly(L-lactide) and its carbonate copolymers. Journal of Microencapsulation. 20(6):731-43, 2003.

U.S. Patent Documents

| | | |
|---|---|---|
| 4,605,738 | Aug. 12, 1986 | Kamata, et al. |
| 4,622,325 | Nov. 11, 1986 | Fujii, et al. |
| 4,631,342 | Dec. 23, 1986 | Umemoto, et al. |
| 4,650,801 | Mar. 17, 1987 | Fujii, et al. |
| 4,652,570 | Mar. 24, 1987 | Fujii, et al. |
| 4,704,393 | Nov. 3, 1987 | Wakabayashi, et al. |
| 4,719,213 | Jan. 12, 1988 | Fujii, et al. |
| 4,757,139 | Jul. 12, 1988 | Kawaguchi, et al. |
| 4,810,790 | Mar. 7, 1989 | Yamamoto, et al. |
| 4,864,021 | Sep. 5, 1989 | Fujii |
| 4,914,105 | Apr. 3, 1990 | Fujii, et al. |
| 4,983,609 | Jan. 8, 1991 | Fujii |
| 5,032,680 | Jul. 16, 1991 | Kawai, et al. |
| 5,047,521 | Sep. 10, 1991 | Fujii, et al. |
| 5,049,551 | Sep. 17, 1991 | Koda, et al. |
| 5,077,055 | Dec. 31, 1991 | Muller, et al. |
| 5,089,503 | Feb. 18, 1992 | Johnson |
| 5,116,600 | May 26, 1992 | Fujii, et al. |
| 5,457,187 | Oct. 10, 1995 | Gmeiner, et al. |
| 5,496,810 | Mar. 5, 1996 | Schwartz |
| 5,610,160 | Mar. 11, 1997 | Sloan, et al. |
| 5,614,505 | Mar. 25, 1997 | Gmeiner, et al. |
| 5,627,187 | May 6, 1997 | Katz |
| 5,663,321 | Sep. 2, 1997 | Gmeiner, et al. |
| 5,676,973 | Oct. 14, 1997 | Levin |
| 5,808,049 | Sep. 15, 1998 | Yamazaki, et al. |
| 5,817,666 | Oct. 6, 1998 | Katz |
| 5,843,917 | Dec. 1, 1998 | Boyd, et al. |
| 6,403,569 | Jun. 11, 2002 | Achterrath |
| 6,670,335 | Dec. 30, 2003 | Singh, et al. |
| 6,794,370 | Sep. 21, 2004 | Achterrath |

Foreign Patent Documents

| | | |
|---|---|---|
| 1246204 | October 1989 | JP. |
| 2091009 | March 1990 | JP. |

What is claimed is:

1. The prodrug of claim 1 having the structural formula
polysaccharide-R-Z, wherein
the polysaccharide is a galactose-containing polysaccharide, Z comprises a therapeutic parent compound and R comprises the covalent bond connecting Z and the galactose-containing polysaccharide.

2. The prodrug of claim 1 wherein the galactose-containing polysaccharide comprises a plurality of galactose residues.

3. The prodrug of claim 2 wherein the polysaccharide has a molecular weight of about 1 Da to about 10⁷ Da.

4. The prodrug of claim 1 wherein the parent therapeutic compound comprises an atom available for linkage to the polysaccharide, the atom being selected from the group consisting of oxygen, nitrogen or sulfur.

5. The prodrug of claim 1 wherein R comprises a linkage selected from the group consisting of —(CH₂)ₙ—, —CO—, —CO(CH₂)ₙ—, and —CO(CH₂)ₙ—CO— wherein n is from 1 to 5.

6. The prodrug of claim 1 wherein R comprises a linkage selected from the group consisting of —NH(CH₂)ₙ—, —CONH—, —CONR₂—, —O—NH—, wherein n is from 1 to 5, and R₂ is a branched, unbranched or cyclic aliphatic or aromatic having from 2 to 20 carbon atoms.

7. The prodrug of claim 1, wherein R comprises an ester, an ether, an amide, an amine, an acylamine, a hydroxylamine, a thioether or thioester.

8. The prodrug of claim 1 wherein the galactose-containing polysaccharide, or a hydrolyzed galactose-containing part thereof binds to galectin-3′.

9. The prodrug of claim 1 having the structure shown in FIG. 1.

10. The prodrug of claim 8 wherein the galactose-containing part thereof comprises at least one galactose moiety capable of binding to galectin-3.

11. The prodrug of claim 7 or 8 wherein the galactose-containing part comprises at least one galactose to which the parent therapeutic compound is bound.

12. The prodrug of claim 1 wherein the parent therapeutic compound is an anticancer compound selected from the group consisting of 5-FU.

13. The prodrug of claim 11 or 12 wherein the parent therapeutic compound is 5-FU.

14. The prodrug of claim 1 wherein the prodrug is orally administered.

15. The prodrug of claim 14 wherein the prodrug reaches the colon in a substantially intact form.

16. The prodrug of claim 1 wherein the prodrug binds to galectin-3.

17. A pharmaceutical composition comprising an effective amount of a prodrug having the structural formula polysaccharide-R-Z wherein,
a) the polysaccharide is a galactose-containing polysaccharide;
b) Z is 5-FU, and
optionally comprising a pharmaceutically acceptable carrier, filler and/or adjuvant.

18. The pharmaceutical composition of claim 17 in a form suitable for oral administration.

19. The pharmaceutical composition of claim 18 wherein the form suitable for oral administration is a tablet, capsule, geltab, caplet, liquid, suspension, drops, gel, syrup, slurry, tincture, lozenge, gum, or any other suitable oral formulation in any appropriate vehicle to be used for cancer patients.

20. A method for preparing a prodrug having the structural formula polysaccharide-R-Z, comprising the steps of,
a) hydrolyzing pectin, guar gum and carob bean gum in alkali at a pH from about 9 to about 10;
b) hydrolyzing the product of step a) in acid at a pH from about 3 to about 5;
c) purifying the polysaccharide, and
d) reacting the polysaccharide with parent therapeutic compound Z, thereby forming covalent bond R comprising either an ester, an ether, an amide, an amine, a hydroxylamine, a thioester, or a thioether, and
and wherein the polysaccharide is a galactose-containing polysaccharide

21. The method of claim 20 further comprising the step of modifying a hydroxyl group in the galactose-containing polysaccharide by adding a functional group selected from the group consisting of ester, an ether, a carboxylic acid, an acyl chloride, or an amide.

22. The method of claim 20 or 21 further comprising the step of modifying an O, N or S atom of the parent therapeutic compound by adding a functional group selected from the group consisting of ester, an ether, a carboxylic acid, an acyl chloride, or an amide.

23. The method of claim 20 or 21 wherein the galactose-containing polysaccharide is a molecular weight of approximately 10⁵ Da to approximately 10⁷ Da.

24. The method of claim 20 or 21 wherein the prodrug is substantially unhydrolyzed while passing through the upper GI tract.

25. The method of claim 20 or 21 wherein the prodrug is hydrolyzed while passing through the colon.

26. The method of claim 20 or 21 wherein Z is 5-FU.

27. The method of claim 25 wherein Z is 5-FU.

28. A prodrug having the structural formula
polysaccharide-R-Z, wherein
the polysaccharide is a naturally occurring galactose-containing polysaccharide,
Z is 5-FU, and
R comprises a carboxyl, ester, ether, or an amide.

29. The prodrug of claim 28 wherein R is an ester or an amide.
